(19) United States
(12) Patent Application Publication
(10) Pub. No.: US 2022/0068440 A1
(43) Pub. Date: Mar. 3, 2022

(54) SYSTEM AND METHOD FOR PREDICTING QUALITY OF A CHEMICAL COMPOUND AND/OR OF A FORMULATION THEREOF AS A PRODUCT OF A PRODUCTION PROCESS

(71) Applicant: Bayer Aktiengesellschaft, Leverkusen (DE)

(72) Inventors: Thomas MRZIGLOD, Bergisch Gladbach (DE); Lynn WÜRTH, Köln (DE); Tom MAES, Leverkusen (DE); Kai Christopher WELLNER, Nievenheim (DE); Stephan TOSCH, Sprockhövel (DE); Christian BOCK, Köln (DE)

(73) Assignee: Bayer Aktiengesellschaft, Leverkusen (DE)

(21) Appl. No.: 17/276,020

(22) PCT Filed: Sep. 17, 2019
(86) PCT No.: PCT/EP2019/074792

§ 371 Date: Mar. 12, 2021

(30) Foreign Application Priority Data
Sep. 18, 2018 (EP) 18195266.4

(51) Int. Cl. G16C 20/30; G06F 30/27; G16C 20/70; G16C 20/10

(52) U.S. Cl. CPC G16C 20/30; G16C 20/10; G16C 20/70; G06F 30/27

(57) ABSTRACT

The present disclosure generally relates to the field of model-based quality prediction of a chemical compound-and/or of a formulation thereof as the outcome of a production process comprising more than one sub-process. It further relates to a solution for root cause analysis of variations of one or more quality attributes of said product or formulation thereof.

### CROSS REFERENCE TO RELATED APPLICATIONS

This application is a national stage application under 35 U.S.C. § 371 of International Application No. PCT/EP2019/074792, filed internationally on Sep. 17, 2019, which claims the benefit of European Application No. 18195266.4, filed Sep. 18, 2018.

### FIELD OF THE DISCLOSURE

The present disclosure generally relates to the field of model-based quality prediction of a chemical compound-and/or of a formulation thereof as the outcome of a production process comprising more than one sub-process. It further relates to a solution for root cause analysis of variations of one or more quality attributes of said product or formulation thereof.

### BACKGROUND

Chemical compound or product refers to any compound produced by an organic or biochemical process. It may be a small or large molecule, such as polymers, polysaccharides, polypeptides. Such a production process may comprise not only the step(s) leading to the compound itself but also its cleaning and formulation steps as well as cleaning of the production plant, feeding paths and/or recycling steps. Each step of the production process and/or parameters thereof may influence the quality attributes of the end-product.

Product quality is a key issue for chemical products in particular in the pharmaceutical field where it is strongly regulated and audited. Process control schemes within a production plant use a number of individual single-input single-output (SISO) control loops to control process variables (also called parameters) such as temperature, agitation speed, pressure, dissolved oxygen, pH, etc., to specific set points. Regulatory constraints have reinforced this traditional method of SISO control methodologies for reactors, bioreactors and other apparatuses, making more data available for analysis. Production process knowledge has become a mix of process understanding acquired by experience, that is expert knowledge, and a growing amount of historical process data hiding hints for identification of uncovered causes for process variations.

For variations in product quality, root cause analysis is a very challenging task, some causes may be suspected, others remaining hidden. Model-based quality prediction for a single step is known (Review of S. Agatonovic-Kustrin et al., Basic concepts of artificial neural network (ANN) modeling and its application in pharmaceutical research, J-Pharm. & Biochem. Anal. 22(2000), 717-727). A multistep production process is more complex: some steps may impact one or more product quality attributes whereas others may have no or little impact thereon. Similarly, some process variables in a particular step may not, little or strongly influence the product quality attribute(s).

Among others, quality of starting material(s), reactant(s) and intermediate(s), occurring of by-product(s), use of recycling steps, process interruptions e.g. for cleaning, inconsistent or missing measurement data, missing metadata—for example time of sampling or information on batch number and/or material, temporary storage of intermediates, renaming and mixing of batches—may complicate the root cause analysis of such variations.

### SUMMARY OF THE DISCLOSURE

In many cases product quality control relies on collecting and analyzing samples in one or more experiments along the production process and/or at the end of the production path. Such sampling and analysis are time consuming, expensive and does not allow prompt evaluation of the current quality of a running or just finished batch or campaign.

Therefore, there is a need for a solution able to quickly provide reliable information on product quality for better deviation management in a timely manner Quick information would speed decision on downtime and runtime, hopefully shortening the latter. Further there is a need for a solution allowing quick and reliable root cause analysis of process steps and parameters thereof impacting quality of products for better targeted control and/or improvement of the production process.

Described herein is a method and a system capable of predicting values for product quality attributes of a chemical compound or of a formulation thereof as an outcome of a multistep production process, wherein the whole process and/or process steps are characterized by process parameters. It is achieved by executing a multivariate data analysis of the process data in a quality-prediction model, which specifies or represents mathematical relationships between quality attributes and process parameters of the production process and/or of sub-processes thereof. Used quality-prediction model is obtained by mathematical modelling of historical process data, most preferred using neural network model(s) in combination with process knowledge gained by the process experts over time. Here, the combination with process knowledge can be the well-considered choice of appropriate input parameters or key performance indicators (defined combination of process parameters) to the model that represent the underlying physical process in a manner that allows for quality prediction as well as knowledge of the chemical or physical behavior or properties of an apparatus or subsystem.

Prediction is usually run on a finished batch but may be conducted on a running batch provided real time data have been collected at the time of the prediction.

Typical quality attributes for end products are as a matter of example without being limited thereto:
overall process yield, concentration of main and/or side products in a reactor or in a formulation, optimal batch run times (such as reaction and/or distillation steps, cut over in chromatography),
viscosity, loss on drying, crystallization, particle size distribution, tablet hardness, Active Pharmaceutical Ingredient (API) or more generally compound release or release rate of active ingredients in a formulation, etc. . . . .

The present disclosure was shown to be applicable for chemical and/or biochemical production processes comprising one or more steps for the production of small or large molecules such as polymers, polysaccharides, or polypeptides as well as of mixtures thereof. Said production processes may comprise reaction steps, cleaning, recycling and/or formulation steps. Formulation may be liquid or solid dosage forms such as powders, tablets, etc.

The present disclosure was shown to be able to increase process understanding—confirming or refuting assumptions and exploring unsuspected correlations between process parameters and quality attributes.

According to some embodiments, the method and system of the invention are of particular interest for production processes wherein only a limited number of product quality measurements is practicable, e.g. one analysis per batch/lot or periodically during continuous production steps.

Batch, period of time for a continuous process are together referred to as prediction instance.

In some embodiments of the method of the invention, the predicted value for a product quality attribute of one or more final and/or intermediate products of a production process is obtained for a prediction instance by:
i. Providing at least one quality-prediction model of the production process, wherein said quality-prediction model specifies or represents mathematical relationships between:
one quality attribute of the product to be predicted and
   process parameters of the production process and/or of sub-processes thereof
ii. Receiving process time series data for a new prediction instance,
iii. Calculating derived quantities as required by the quality-prediction model,
iv. Executing the quality-prediction model by feeding the process time series data and/or the calculated derivatives of iii., generating prediction results for the quality attribute,
v. Output the prediction results for the quality attribute as a single quality value or as a curve as the case may be.

In a preferred embodiment several quality-prediction models are provided, each one calculating one quality attribute.

The quality-prediction model comprises at least one data-based prediction model: The data-based prediction model(s) are typically obtained by modelling historical process time series data.

Historical process time series data are time series of process parameter values collected in previous batches or time periods as well as their respective values for quality attributes as measured.

A data-based prediction model may be a neural network or multivariate models such as partial least squares regression (PLS).

In a preferred embodiment the data-based prediction model comprises several data-based prediction models.

In a first embodiment each data-based prediction model may be trained on process parameter to provide intermediate variables for which physical or empirical correlations are known or available. Generated data-based prediction model are then combined using physical or empirical correlations in a hybrid model.

Preferred data-based prediction model are neural networks for their ability to model arbitrary mathematical functions (i.e. also non-linear behavior) in a very efficient manner.

Most preferred are neural networks with one input layer, one hidden layer and one output layer (as described by F. Barmann, F. Biergler-König: On a class of efficient learning algorithms for neural networks, Neural Networks, Vol. 5(1), 1992, 139-144, which teaching is integrated per reference). In a particular embodiment, during the training steps of the neural network, the number of nodes in the hidden layer as well as their respective weights are optimized using a mathematical solver implemented in the commercially available NN-Tool (Reference: http://www.nntool.de/Englisch/index_engl.html). The training itself most preferably comprises cross-validation steps (e.g. block-wise, random, every n-data point), wherein part of the available time series data (usually around 10%) is not used in the training steps. After the training this remaining part of data is used to test the predictive strength of the model. The goal of this cross-validation process is to avoid the modeling of random correlation and/or over-fitting of the model.

In a further embodiment the quality-prediction model also comprises one or more mechanistic model(s) for one or more steps, e. g. thermodynamic and/or kinetic model(s). Such mechanistic models are typically fundamental models making use of chemical and/or physical first principles such as heat and mass balance, diffusion, fluid mechanics, chemical reactions etc.

It is most preferred for the quality-prediction model to comprise a combination of data-based, and a mechanistic modelling into a hybrid model. Such hybrid models are more robust as they allow for a certain extent of extrapolation, which pure data-based models do not. Extrapolation means that they are able to produce a trustworthy prediction outside of the convex hull of the data set that they were trained on.

### BRIEF DESCRIPTION OF THE DRAWINGS

The invention will now be described, by way of example only, with reference to the accompanying drawings, in which:

FIG. 1 shows a visualization of the results on a dashboard, according to some embodiments;

FIG. 2 shows a block diagram of an example of hybrid model, according to some embodiments;

FIG. 3 shows a block diagram of an alternative embodiment of a hybrid model, according to some embodiments;

FIG. 4 shows a high-level block diagram of a system for product-quality prediction, according to some embodiments;

FIG. 5 shows a diagram summarizing steps relating to model building and identifying the impact factors, according to some embodiments;

FIG. 6 shows an exemplary production process for a small molecule, according to some embodiments;

FIG. 7 shows agreement for product yield of model-based prediction and laboratory results, according to some embodiments;

FIG. 8 shows process time series data, according to some embodiments; and

FIG. 9 shows predictions made by a method matches the classical laboratory analysis.

### DETAILED DESCRIPTION OF EXEMPLARY EMBODIMENTS

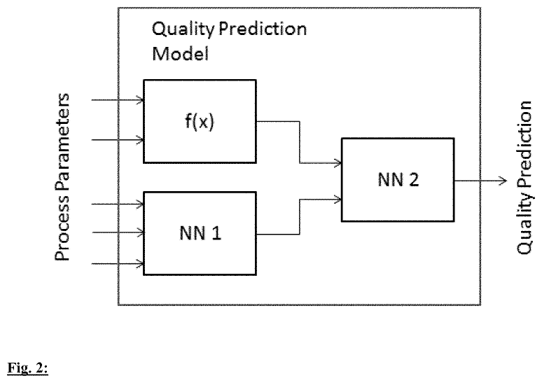

FIG. 2 shows a block diagram of an example of hybrid model, wherein the process parameters are inputted in a first model layer comprising the neural network prediction model NN 1 and the mechanistic model f(x); results calculated by the models of the first layer are inputted into a second neural network model NN2 to calculate the final prediction.

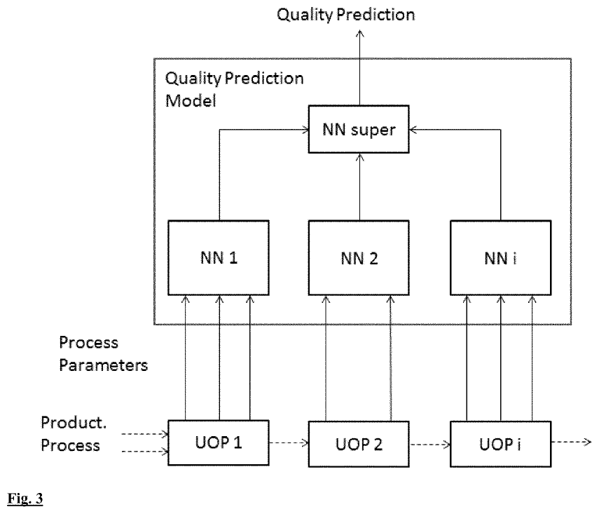

In a particular embodiment each data-based model may describe one production step, the several models being organized in a hybrid model. FIG. 3 shows a block diagram of an alternative embodiment of a hybrid model. The process is conducted in unit operations (UOP), one neural network prediction model is used for each UOP (NN 1, NN 2, NN i); an overarching supervisory model (NN super) is getting inputs from NN1 to NNi and provides final prediction.

Numerous variations and modifications of the quality-prediction model will become apparent to those skilled in the art, said model being built in consideration of the production process at stake.

The quality-prediction model is built by:
a) Receiving a description of a production process as one or more interrelated sub-processes and their respective process parameters,
b) Receiving a quality attribute of the product to be modeled and predicted, wherein said product may be final and/or intermediate product of the production process at stake,
c) Receiving at least one sub-process which is suspected to influence the product quality attribute. Typically, first information is provided using expert knowledge.
d) For each of the sub-processes of c), receiving process parameters suspected to influence the quality attribute.
e) In a particular embodiment receiving for each process parameter of d) derived quantity(ies) suspected to influence the product quality attribute of b).

Typically, first information for step c), d) and/or e) is expert knowledge introduced by an operator or received from a database. Introduction of this expert knowledge is also referred to as supervised training or supervised learning. In the iteration loop (step k) further process parameters and/or derived quantities may be included into the analysis.
f) Receiving historical process time series data of production processes as defined in steps a) to d), comprising measured data for the process parameters of a) over a time period and value for the quality attribute of the product of b),
g) Calculating the values of the derived quantities of step e) for all the time series data if needed,
h) It is preferred to eliminate derived quantities of step g) and/or process parameters that contain redundant information, noise, or other non-relevant information e.g. using cross-correlation matrix or Principle Component Analysis (PCA), and/or expert knowledge where appropriate. As a result a meaningful subset of process parameters and/or derived quantities is provided.
i) Building quality-prediction model propositions in:
   a. Training of one or more data-based prediction models using the historical time series data and/or the values of the derived quantities of g), preferably using the subset of process parameters and/or derived quantities of step h). It may be helpful to use different data-based prediction models for the sub-processes and combine them at a later stage;
   b. In a preferred embodiment, for at least one of the data-based models of step a. a reduced data-based prediction model proposition is provided by:
      calculating the influence of each process parameters and/or derived quantities on the values of the quality attribute and performing a goodness of fit analysis;
      reducing the number of process parameters and/or derived quantities by identifying and removing the parameters and/or derived quantities with the lowest influence on the value of the quality attribute so a reduced data-based prediction model proposition is obtained and saved together with its goodness of fit;
      iterating steps a. and b. to obtain a set of reduced data-based prediction model propositions;
      selecting the most appropriate reduced data-based prediction model proposition in consideration of the goodness of fit, preferably in combination with the physical and/or mechanistic coherence of the data-based prediction model proposition;
   c. In a further preferred embodiment building mechanistic model(s) for one or more steps;
   d. Combining the data-based prediction model(s) of a., preferably the reduced data-based prediction model proposition of step b., and the mechanistic model(s) of step c. into a hybrid quality-prediction model;
   e. Using the hybrid quality-prediction model of d. calculating for each historical process time series a prediction value for the quality attribute and a goodness of fit to the value for the quality attribute as recorded in the historical process time series; providing and saving the quality-prediction model proposition with its set of influencing process parameters and/or derived quantities and most preferred a value characterizing the degree of their respective influence on the quality attribute at stake, as well as its goodness of fit;
   f. Iterating steps i) a. to i) e. by systematically leaving out parameters and/or derived quantities; a set of reduced hybrid quality-prediction model propositions is obtained;
j) Receiving the quality-prediction model propositions of step i); selecting the model proposition leading to the best goodness of fit by means of expert knowledge in view of its physical and/or mechanistic coherence. Expert knowledge also preferably comprises considerations on random correlation and/or over-fitting of the model;
k) Iterating a-j) introducing or deleting one or more of the production sub-processes, their respective process parameters and/or derived quantities until an acceptable goodness of fit is achieved;
l) As a result providing a final quality-prediction model for the production process, defined by its goodness of fit, a set of most influencing process parameters and/or derived quantities, most preferred together with a value characterizing the degree of respective influence of said process parameters and/or derived quantities on the quality attribute at stake.

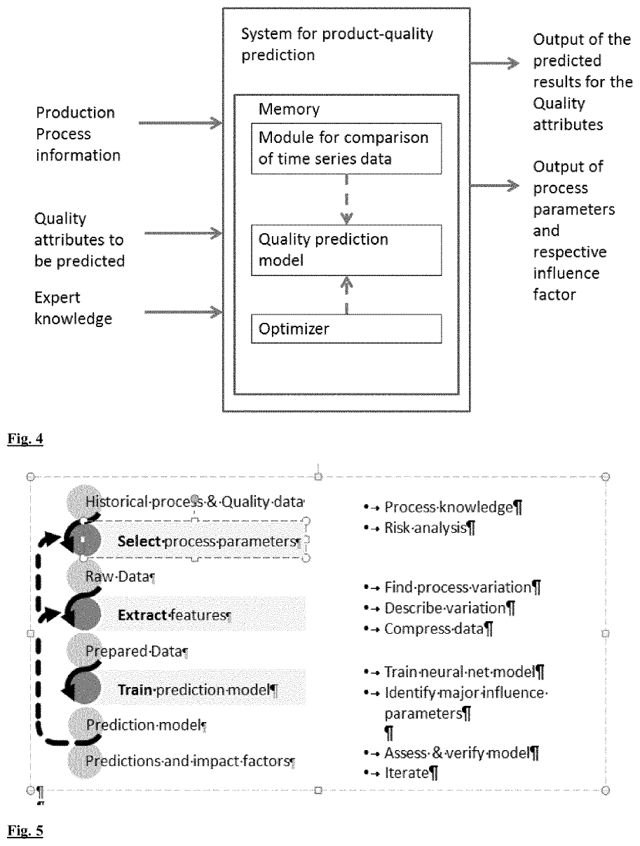

The generation of the quality-prediction model is summarized in FIG. 5.

Typical sub-processes suspected to influence product quality attributes are chemical/biochemical reaction in a (bio)reactor, purification steps—such as chromatography, distillation, etc, recycling steps, process interruptions such as cleaning steps, formulation of solids such as granulation, tableting and coating. Numerous combinations of sub-processes will be apparent to those skilled in the art.

Process parameters may be primary (measured parameter) and/or secondary parameters (indirect parameters, e.g. kinetic information). Examples for such process parameters are:
quality attributes of starting material(s) and/or of intermediate(s) generated in a sub-process,
concentrations of starting material(s) and/or intermediate(s), concentration of secondary product(s),
physical parameters such as temperature, pressure,
control parameters such as level, and/or flow control schemes, cascade, feedforward, and/or constraint control schemes,
single values or variation over time as well as a tolerance for parameter variation
Examples for process parameters for cleaning steps are: duration of cleaning, amount and type of cleaning agents applied.
Examples for process parameter for recycling steps are: concentration of material fed back, flow rate (continuous) or amount (batch)
Examples for secondary parameters are: heat flow rate calculated from heat balance (using volumes, flow rates and temperatures), stoichiometry of starting materials, quality attributes from previous batches or previous time intervals for continuous campaigns. These latter allow consideration of time delayed influences of for example recycle streams, residual material in filter(s) and vessel(s)—reactor, columns, etc.

Historical process time series data ideally comprises data for process parameters over a time period (time series) and respective values for quality attributes of final products collected in previous batches (together also referred to as historical process and quality data), most preferred also quality data of starting materials and intermediates are used. It is preferred that historical process time series data comprises as much process parameter and quality data from previous batches or for continuous processes previous periods of time as possible. In considering these data sets it is advisable to consider their validity in view of the production process to be modelled. As a matter of example, a piece of historical process time series may refer to a batch, wherein intermediate steps were apportioned, or several intermediates steps were joined for further processing. In such cases, the relation between the values of the quality attributes as measured may relate to a whole batch or to a portion thereof, also quality attributes of intermediates may be relevant.

In a particular embodiment of the method of the invention a goodness of fit for the training of the data-based model is conducted for each piece of historical process time series. For this purpose, it is preferred that the historical process time series data are provided in form of a spreadsheet. For each piece of time series, a model proposition leading to the best goodness of fit is calculated together with a quantification of the uncertainty of the model as well as a quantification of how much each input is contributing to the output uncertainty (sensitivity analysis). It is preferred that these quantifications are displayed to an expert via a user interface. The expert is required to confirm the validity of the input by means of expert knowledge and/or the quantifications mentioned above. The expert shall decide if the piece of historical process time series shall be considered or rejected for training of the data-based model. In other words, it is preferred that historical process time series (input) is controlled for goodness of fit for training the data-based model. It is most preferred that such control is conducted in a semi-automatic way, that is that expert knowledge is considered in validating the input.

Derived quantities may be for example min value, max value, average value, standard deviation, a quantity at a specific point in time, max- or min value of time derivatives or integrals or a combination thereof. In a particular embodiment, derived quantities may be the result of a multivariate analysis, for example loading vectors.

Adequate derived quantities may be identified by inspection of historical time series data from different batches and/or using mathematical methods like Principal Component Analysis (PCA) or Partial Least Squares Regression (PLS).

In a particular embodiment for the identification of the derived quantities that contain redundant information, noise, or other non-relevant information (step h), a cross-correlation matrix of all these quantities is calculated and evaluated. Evaluated means, that with the help of the cross-correlations some of the statistical quantities are excluded from further analysis. For this highly iterative process, experience and expert knowledge are applied to select correlating parameters to exclude. The removal of the redundant highly correlated statistical quantities is advantageous to reduce the noise on the data and improve the predictive strength of the resulting model.

In a particular embodiment, the iteration step k) may be conducted via an optimizer, e.g. varying one or more of the production steps, process parameters thereof and/or derived quantities and assessing the resulting model outputs according to goodness of fit.

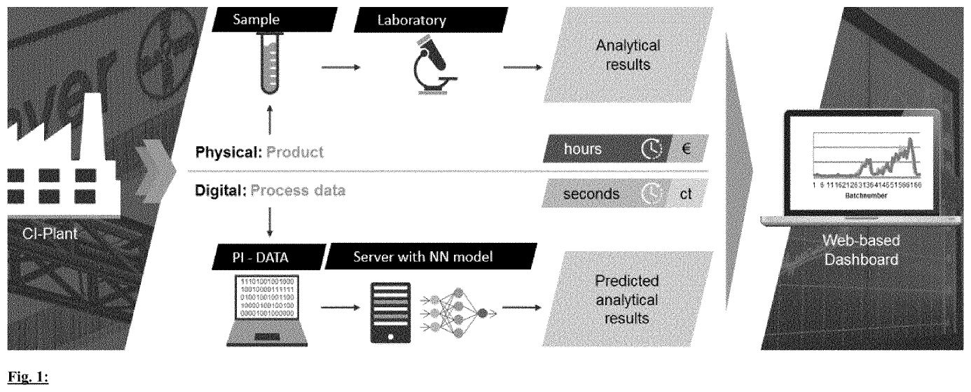

It is preferred to output for each object of the prediction, the predicted value of the quality attribute, a list of the identified critical quality influencing process parameters and/or derived quantities thereof (together also referred to as impact factors), most preferred respectively with a value characterizing the degree of influence of said process parameter/derived quantity on the quality attribute at stake. For better understanding it is most preferred to provide a visualization of the results on a dashboard, most preferred in a web-based dashboard (FIG. 1).

According to some embodiments, the quality-prediction model generated by the method of the invention may be used:
To provide prediction values for a set of quality attributes for a new prediction instance in real time or retrospectively,
To provide a list of process parameter or derived quantities thereof influencing the variation of the quality attributes of interest,
to define limits for a set of process variables or parameters derived from process variables (design space) to keep the product quality in a predefined spectrum,
to output setpoints for process variables during different production steps and control the process with calculated set points,

in particular cases to simulate quality results of possible process changes, e.g. by receiving time series data for a fictive batch in the prediction steps,

Typically, the method mentioned above is run in a system for product-quality prediction comprising elements configured to conduct the method steps mentioned above. In one embodiment the quality-prediction model is stored in a model module. Receiving steps may be achieved by interfacing the model module with respective databases to enable receiving of expert knowledge and/or data, in particular for real time feeding of data allowing real time prediction. Also, a user interface may be used in particular for introduction of expert knowledge, such as quality attributes, process information and/or process knowledge—sub-process and/or parameters suspected to influence the quality attributes. Output is generally displayed on a user interface, preferably in graphical form. Most preferred a dashboard is used for easy navigation between the results in particular a web-based dashboard (for example FIG. 1,7,8).

In a particular embodiment of the method of the invention, new time series data for a production process are used for continuous improvement of the quality-prediction model representing said process. In such an embodiment the system of the invention may comprise a module for comparison of time series data configured to recognize new or unknown process states and trigger an automatic retraining of the quality-prediction model. For this latter purpose the module for comparison of time series data is interfaced with the model module.

According to some embodiments, a further object of the invention is a system for product-quality prediction comprising elements configured to conduct the method steps as described above. A high-level block diagram of such a system for product-quality prediction is shown in FIG. 4 as a matter of example.

FIG. 5 shows a diagram summarizing the steps relating to model building and identifying the impact factors (=influencing process parameters and/or derived quantities). According to some embodiments, an objective of the invention is also a computer program product storing program instructions, wherein the program instructions are executable to perform the steps of the method as mentioned above.

Numerous variations and modifications of the solutions of the invention will become apparent to those skilled in the art once the above disclosure is fully appreciated.

The solution of the invention was used in several examples described below. Usability is not limited thereto.

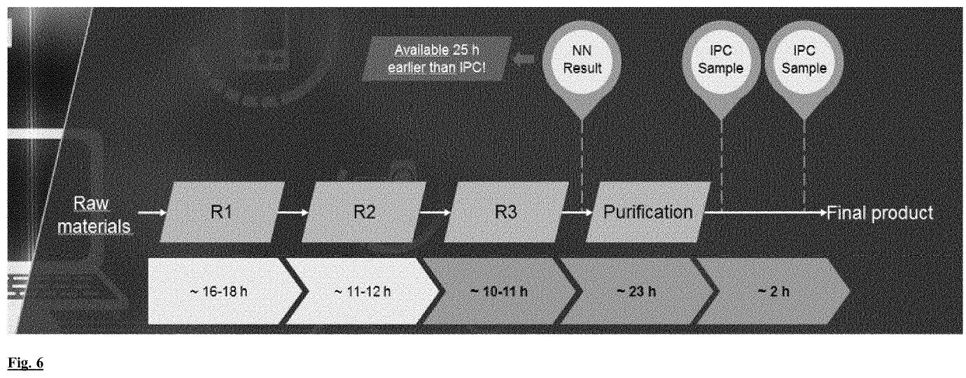

### Example 1—Production of a Small Molecule in a Production Process with Intermediates as Shown in the Diagram of FIG. 6, Wherein R1, R2, R3 are Reaction Steps

In the past, variations in product quality lead to a large number of out of specification batches for the production of this chemical compound taken as an example. Among others product yield and concentration of by-products were subjected to unclear variations. Applying the quality prediction methodology enabled to get a grip on the underlying root causes for these quality issues.

The provided quality-prediction model comprised a neural network model for each of the two predicted quality attributes. All process parameters available in the historical time series data were considered in the training in an iterative manner. The predicted quality parameters were the product yield and the concentration of one by-product.

According to some embodiments, the method of the invention was used to predict the product quality prior to the laboratory result, thus giving the operators more time to react to deviations.

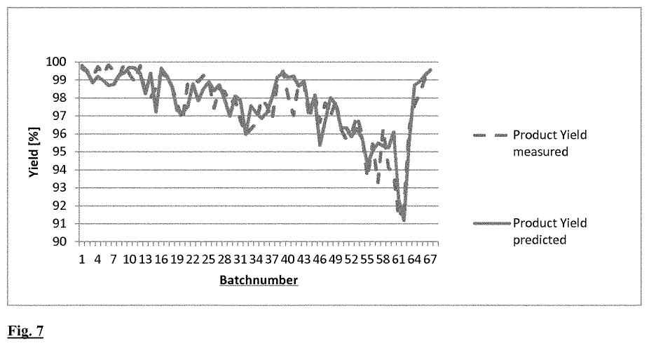

The neural network model was trained using derived quantities calculated from historical process data. Min, Max, Average and slope of Temperature were identified to be relevant for the quality-prediction model. FIG. 7 shows the agreement for the product yield of model-based prediction and laboratory results to a very high degree. Furthermore, the main influencing parameters for both predicted quality parameters were outputted.

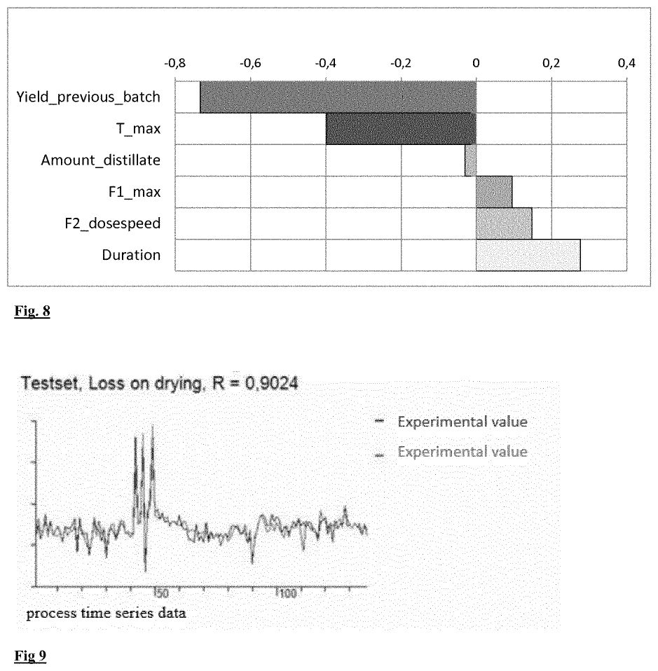

In this case, the two most important influencing factors for the quality of the product (depicted in FIG. 8) were the maximum temperature and the performance of the previous batch. The latter factor gave an indication that undesired back-mixing may have occurred during the production process. Other theories that were raised by the operating personnel, for instance a possible influence of interim cleaning procedures, could by discarded by the analysis.

In a subsequent step, this additionally gained process understanding was used to propose a variety of preventive measures that were implemented and allowed to bring the process back into its normal operating range.

Further the quality prediction model was interfaced to the process historian to enable an online, real-time prediction. For this purpose, the model was executed periodically on a server that has access to the real-time process data (via the process historian). As soon as process data of a new batch were available a new quality prediction was calculated and displayed in a web-based dashboard (as summarized in FIG. 1). The dashboard shows past and current quality predictions as well as their corresponding laboratory results (provided they are already available).

In the last production campaign, the model-based quality prediction was available 25 hours prior to the laboratory results on average (see FIG. 6). Given a batch run time of 10-11 hours for the quality-critical reaction step, this allowed the operators to better control the process and manipulate appropriate process parameters in time. Production of out-of-specification batches due to the long time period between sampling and laboratory result could be avoided.

### Example 2—Production of API (Active Pharmaceutical Ingredient) Quality Release Data in a Bio-Production Process

In Example 2 quality prediction of an Active Pharmaceutical Ingredient (API) was performed at the final stage of a bio-production process.

The product quality of the API in the considered bio-production process is defined by several quality attributes, which are specified in the registration of the API. These include the concentration of the API, as well as of any impurities resulting from side reactions; the registered concentration ranges must be strictly observed. Furthermore, other parameters, such as the water content, must also be determined and meet the specification at the end of each batch. These quality attributes of the final API product were defined as the output variables of the quality prediction models. In this case study, each quality attribute was described by a specific neural network (NN) model (also referred to as NN-model).

Information available from In Process Control, analytical measurements taken further upstream in the process, process parameters values measured continuously in the production equipment (e.g. pressures, temperatures, pH, etc.) and quality data for historical campaigns were used for model training as described above. The data had to be merged from several sources of data collection. It was prepared by removing outliers, smoothing if required, and were used for model training of the model. A set of process parameters having the highest influence on each product quality attribute at stake was identified using the method described above. For model training a data set collected over one year of production was used.

For each NN-model predicting a specific quality attribute, a different set of process parameters was identified. This set was used as a preferred set of input data for the prediction of product-quality for a new prediction instance.

Most influencing process parameters were for instance the maximum temperature reached during a specific phase of the batch process or a variation of a process parameter over time described by the mathematical calculation of a derivative at a certain batch stage. For example, to build a prediction model for the water content, several process parameters from the final drying step such as temperature, pressure, drying duration and data from further upstream processing steps were used to describe the characteristic variance having an impact on the remaining water content in the product.

A substantial part of the analytical measurements required for quality release of the product, which are normally determined in the laboratory after the end of the batch, was predicted by the method of the present invention, according to some embodiments. The prediction was of very high quality as verified subsequently during monitoring tests in the real production process.

The good quality of the API for the final stage of the API production process achieved in the present case-study shows opportunities for real-time product release with the help of a NN prediction model. Real-time quality prediction would bring higher efficiency in production lead times, sparing time required to complete product quality tests at the end of production by sampling and running analytical measurements in the laboratory. Currently the product can be released from a quality perspective and sent to further processing steps as e.g. formulation and tableting only after necessary quality analytical measurements are run and quality is confirmed.

Besides potential efficiency gains in the supply chain, the results of the quality prediction modeling were also used to improve process understanding by quantifying the impact different production factors had on process variation.

### Example 3—Quality Prediction of the Release Rate of an Active Ingredient Incorporated in a Polymer Mixture

In a further case study quality prediction for the production process of a medical product, which releases an active ingredient at a controlled rate from a polymer mixture, was achieved.

The production process at stake involves several manufacturing steps. The raw materials consist essentially of the polymer mixture and the active ingredient, for which physical and chemical analysis results were available.

The product quality is mainly characterized by a measurement of the release rate of a statistically representative number of samples in the laboratory. The laboratory measurement for the experimental measurement of this quality attribute is designed to reflect the release of the active ingredient over time during actual usage of the product. This measurement is performed on samples collected at the end of the production process and the measurement result must be above a certain target to fulfill the required specification. Said release rate is the quality attribute to be predicted and is described by a mathematical function fitted to the measured data.

The aim of the case study was to analyze the complex relationships between the raw materials properties, the manufacturing parameters playing a role during the production process, and the release rate of active ingredient of the product at the end of the process.

The input parameters of the model were the raw material quality parameters and the available process parameters (e.g. settings of the production machines and measurements recorded during production).

Data from a relatively high number of batches over several years of production was collected for model training. The data was filtered to generate a complete set of data for all batches considered. As the production process involved different steps and branches, a batch genealogy was built to connect the data points from different process steps, which were linked to a specific product batch at the end of the process.

Due to the complexity of the production process and the interdependencies, the combination of these results together with process expertise was crucial to interpret the results. The resulting model generated by training using the method mentioned above was shown to be able to clearly describe the major variation in the data. A set of input parameters having a significant influence on the release rate was identified.

Hence, the insights from the modeling results were used to identify the process steps and raw material properties of particular interest for further process optimization. According to some embodiments, outputs of the method of the invention were used to design experiments to check real impact of identified most influencing parameters.

Gaining process understanding allowed to explain even small variability occurring in the quality measurements and to further optimize the production process.

### Example 4—Quality Prediction of for a Formulation Process

In a classical formulation process for solid oral dosage forms the raw materials (excipients and active pharmaceutical ingredients) are mixed, granulated, dried, tableted and coated. To ensure a constant product quality that lies within the registered limits, the local quality control and quality assurance organizations rely on in-process controls (IPC) and final product release controls (both of which are carried out by laboratory analyses). This is expensive, time-consuming and may also be a bottleneck for the overall production process.

The solution of the invention was used for a formulation process wherein the product is granulated and subsequently dried in a fluid bed granulator. The quality attribute of interest for this process was the loss on drying value of the granulated product. Drying value of the granulated product is typically obtained by taking a sample and analyzing it in the lab. In the mean-time the granulator waits for clearance. In other words, the formulation can neither be further processed (in case it needs to be re-dried), nor can the granulation unit be used for the next process batch.

The provided quality-prediction model for this use case comprised a neural network model for the quality attribute to be predicted. Historical measurement data from the granulator was used to train the neural network. Prediction was made for new process data. FIG. 9 shows that the predictions made by the method of the invention matches the classical laboratory analysis to a very high degree.

Min, Max, Average and slope of Temperature were identified to be the main influencing parameters for the drying value of the granulated product.

In some embodiments, the method of the invention was used to speed up this release process and save money for costly lab analyses.

1. A computer implemented method for predicting values for product quality attributes of a chemical compound or of a formulation thereof as a final or intermediate product of a production process, said production process comprising more than one sub-processes, wherein said production process and/or sub-processes thereof are characterized by process parameters and corresponding time series data for a prediction instance, said method comprising:
providing at least one quality-prediction model of the production process, wherein said quality-prediction model specifies or represents mathematical relationships between one quality attribute and process parameters of the production process and/or of sub-processes thereof,
receiving process time series data for a new prediction instance,
calculating derived quantities as required by the quality-prediction model(s),
executing the quality-prediction model(s) by feeding the process time series data and/or the derived quantities thereof, generating prediction results for the quality attribute(s), and
outputting the prediction results for the quality attribute(s) as a single quality value or as a curve as the case may be.

2. The computer implemented method of claim 1, wherein several quality-prediction models are provided, each one calculating one quality attribute.

3. The computer implemented method of claim 1, wherein the quality-prediction model is built by:
a) receiving a description of the production process as one or more interrelated sub-processes and their respective process parameters,
b) receiving the quality attribute of the product to be modeled and predicted, wherein said product may be final and/or intermediate product of the production process at stake,
c) receiving at least one production sub-process which is suspected to influence the product quality attribute,
d) for each of the sub-processes of c), receiving process parameters and/or derived quantities thereof suspected to influence the quality attribute,
e) receiving historical process time series data of the production process comprising measured data for the process parameters over a time period and quality data of the product,
f) if needed, calculating values of the derived quantities received in step d) for all the process time series data,
g) building quality-prediction model propositions in:
a. training one or more data-based prediction model using the values of the derived quantities of f) and/or the historical process time series data of e), combining the models of steps a. into a hybrid quality-prediction model proposition, if more than one data-based prediction models are used,
b. calculating for each historical process time series a prediction value for the quality attribute using the quality-prediction model proposition of b., a goodness of fit and providing several prediction model propositions with a set of influencing process parameters and/or derived quantities by iterating steps g)a. to g)e. deleting parameters and/or derived quantities,
h) Selecting the model proposition leading to the best goodness of fit identified by means of expert knowledge in view of its physical and/or mechanistic coherence,
i) Iterating a-h) introducing or deleting one or more of the production steps, process parameters and/or derived quantities thereof until acceptable goodness of fit is achieved,
j) As a result, providing the final quality-prediction model, with a goodness of fit, a set of influencing process parameters and/or derived quantities.

4. The computer implemented method of claim 3, wherein the quality-prediction model comprises at least one data-based model for one or more of the sub-processes.

5. The computer implemented method of claim 4 wherein the data-based model is a neural network.

6. The computer implemented method of claim 3, wherein quality attributes from products of sub-processes are received as process parameters and/or derived quantities suspected to influence the quality attribute of the product to be predicted.

7. The computer implemented method of claim 3, wherein in an intermediate step f′) between f) and g) derived quantities of step f) and/or process parameters that contain redundant information, noise, or other non-relevant information are identified and eliminated.

8. The computer implemented method of claim 7, wherein for step f′) a cross-correlation matrix or Principle Component Analysis is used.

9. The computer implemented method of claim 3, wherein one or more mechanistic model(s) for one or more steps are built and combined with the one or more data-based prediction models into a hybrid model.

10. The computer implemented method of claim 3, wherein in step g)c. the quality-prediction model also calculates a value characterizing a degree of respective influence of process parameters and/or derived quantities on the quality attribute at stake.

11. The computer implemented method of claim 10, wherein the value characterizing the degree of respective influence of process parameters and/or derived quantities on the quality attribute is used to eliminate a least influencing process parameters and/or derived quantities from the quality-prediction model.

12. The computer implemented method of claim 10 wherein the value characterizing the degree of respective influence of process parameters and/or derived quantities on the quality attribute is used to select the process parameter or derived quantities most influencing the quality attributes of interest and wherein said selection optionally with the value characterizing their degree of influence is outputted and/or said selection is used for process control.

13. The computer implemented method of claim 3, wherein the prediction values for the quality attributes for a new prediction instance is calculated in real time and used for product release and/or process control.

14. The method of claim 1, wherein the product is selected from a list comprising polymers, polysaccharides, or polypeptides and or mixtures thereof.

15. A computing system for product-quality prediction comprising elements configured to:
provide at least one quality-prediction model of the production process, wherein said quality-prediction model specifies or represents mathematical relationships between one quality attribute and process parameters of the production process and/or of sub-processes thereof,
receive process time series data for a new prediction instance,
calculate derived quantities as required by the quality-prediction model(s),
execute the quality-prediction model(s) by feeding the process time series data and/or the derived quantities thereof, generating prediction results for the quality attribute(s), and
output the prediction results for the quality attribute(s) as a single quality value or as a curve as the case may be.

16. A non-transitory computer readable storage medium storing one or more programs, the one or more programs comprising instructions, which when executed by an electronic device, cause the device to to:
provide at least one quality-prediction model of the production process, wherein said quality-prediction model specifies or represents mathematical relationships between one quality attribute and process parameters of the production process and/or of sub-processes thereof,
receive process time series data for a new prediction instance,
calculate derived quantities as required by the quality-prediction model(s),
execute the quality-prediction model(s) by feeding the process time series data and/or the derived quantities thereof, generating prediction results for the quality attribute(s), and
output the prediction results for the quality attribute(s) as a single quality value or as a curve as the case may be.
